SEQUENCE LISTING (2 sequences)

SEQ ID NO: 1 - DNA, 2940 bp, Solanum lycopersicum
  source 1..2940: mol_type = other DNA
atggctgaagctttccttcaaattatgttagagaatctgacttgtttcatccaaggggaacttggattgattcttggttttaaggatgagttcgaaaagcttcaaagcacgtttactacaatccaagctgtggtacaagatgctcagttgaagcaattgaaggacaaggcaattgaaaattggttgcagaaactcaatggtgctgcatatgaagctgatgacatcttggacgaatgtaaaactgaggcaccaattatacagaagaagaataaatatgggtgttatcatccaaacgttatcactttccgtcgcaagattgggaaaaggatgaaaaagattatggagaaactagatgcaattgcagcggaacgaattaagtttcatttggatgaaaggactatagagagacaagttgctacacgccaaacaggttttgttttaaatgaaccacaagtttatggaagagacaaagataaggatgagatagtgaaaatcctgataaacaatgcccaaacactttcagtcctcccaatacttggtatggggggactaggaaagacgacccttgcccaaatggtcttcaatgatcagagagtaattgaacatttccatcccaaaatatggatttgtgtctcggaagattttaatgaaaagaggttgataaagaaaattgtagaatctattgaagaaaagtcacttggtgacatggacttggctccacttcaaaagaagcttcaggacttgctgaatggaaaaaaatatttgcttgtcttagatgatgtttggaatgaagatcaagataagtgggctaagttaagacaagtcttgaaggctggagcaagtggtgcttatgttctaaccactacccgtcttgaaaaggttggatcaatcatggggacattgcaaccatatgaattgtcaaatttgtctcaagaagattgttggttgttgttcatgcaatgtgcatttgggcaccaagaagaaatgaatcttaatctagtggctatcggaaaggtgattgtgaaaaaatgtggtggtgtgcctctagcagctaaaactcttggaggtattttgcgcttcaagagagaagaaagacagtgggaacatgtgagagatagtgagatttggaatttacctcaagatgaaagttctattctgcctgccctgagacttagttaccatcaccttccacttgatttgagacaatgcttttcatattgtgcagtattcccaaaggataccaaaatggaaaaggaaaatctaatctctctctggatggcacatggttttcttttatcaaaaggaaacttggagctagaggatgtaggtaatgaagtatggaatgaattatacttgaggtcttttttccaagagattgaagttcaatatgatcgaacttatttcaagatgcatgatctcattcatgatttggcaacatctctattttcagcaagcacatcaagcagcaatatccgagaaataaatgtagaaggttacctacatatgatgtcgattggtttcgcaaaagtggtgtcttcttactctcctcctcacttgcaaaagtttgtctcattgagggttcttaatctaagttccatgggacttaagcagttaccgtcctccattggagatctagtacatttaagatacttgaacctctctctcaataacatgcgtactcttccaaagcagttatgcaagcttcaaaatctgcagactcttaatgtagagtattgctggtcactttgttgttttccaaaagaaacaagtaaacttggtagtctccgaaatctcttacttgatggttgcgatggattggattctatgccaccaaggataggatctttgacatgccttaagactctaagtttatttgttattggcgagagaaaagattctctacttggtgaattacgaaacctgaatctgtatgggtcaattgaaatcacgcatcttgagagagtgaagaatgatagggatgcaaaagaagccaatttatctgcaaaagaaaatctgcattctttaagcatgagatgggaaggaccacatagatatgaatcagaagaagttgaagtgcttgaatccctcaaaccacactccaatgtgacttgtttaacaatcactggcttcagaggaatccgtctcccagagtggatgaatcactcagttttgaaaaatgttgtctctattgcaattagaggttgtgaaaactgctcatgcttaccaccgtttggtgatctgccttgtctagaaagtctagagttacggagtgggtctgcggaagtggagtatgttgaagattctggattcccaacaagaagaaggtttccatctatgagaaaacttactatagaaaattttgataatctgaaaggattgctgaaagaggcaggagaagagcaattccccgtgcttgaagagttgacaattagatgttgtcctgtgtttgttattccgaccctttcttctgtcaagaaattggtagttcatgggaacaagtcagatgcaatagttttgaggtccatatataatcttagggctcttacttccctcaacattagccataacttcacagctacttcgctcccagaagagatgttcaaaagccttgcaaatctcaaatacttggaaatcgctttcatctccaatctcaaagagctgccaaacagcctggctagtctcaatgctttgaagcatctgtttattaattgttgttttgcactagagagtctccccgaggaagcggtgaaaggtttaacttcactcacacagttatccataacatactgtaagaggctaaaatgtttaccagagggattgcagcaactaacaaatttatcagttaggtattgtccaacactggccaagcgatgtgagaagggaataggacaagactggtacaaaattgctcacattcctcatctgctgattactgattag

SEQ ID NO: 2 - protein, 979 aa, Solanum lycopersicum
MAEAFLQIMLENLTCFIQGELGLILGFKDEFEKLQSTFTTIQAVVQDAQLKQLKDKAIENWLQKLNGAAYEADDILDECKTEAPIIQKKNKYGCYHPNVITFRRKIGKRMKKIMEKLDAIAAERIKFHLDERTIERQVATRQTGFVLNEPQVYGRDKDKDEIVKILINNAQTLSVLPILGMGGLGKTTLAQMVFNDQRVIEHFHPKIWICVSEDFNEKRLIKKIVESIEEKSLGDMDLAPLQKKLQDLLNGKKYLLVLDDVWNEDQDKWAKLRQVLKAGASGAYVLTTTRLEKVGSIMGTLQPYELSNLSQEDCWLLFMQCAFGHQEEMNLNLVAIGKVIVKKCGGVPLAAKTLGGILRFKREERQWEHVRDSEIWNLPQDESSILPALRLSYHHLPLDLRQCFSYCAVFPKDTKMEKENLISLWMAHGFLLSKGNLELEDVGNEVWNELYLRSFFQEIEVQYDRTYFKMHDLIHDLATSLFSASTSSSNIREINVEGYLHMMSIGFAKVVSSYSPPHLQKFVSLRVLNLSSMGLKQLPSSIGDLVHLRYLNLSLNNMRTLPKQLCKLQNLQTLNVEYCWSLCCFPKETSKLGSLRNLLLDGCDGLDSMPPRIGSLTCLKTLSLFVIGERKDSLLGELRNLNLYGSIEITHLERVKNDRDAKEANLSAKENLHSLSMRWEGPHRYESEEVEVLESLKPHSNVTCLTITGFRGIRLPEWMNHSVLKNVVSIAIRGCENCSCLPPFGDLPCLESLELRSGSAEVEYVEDSGFPTRRRFPSMRKLTIENFDNLKGLLKEAGEEQFPVLEELTIRCCPVFVIPTLSSVKKLVVHGNKSDAIVLRSIYNLRALTSLNISHNFTATSLPEEMFKSLANLKYLEIAFISNLKELPNSLASLNALKHLFINCCFALESLPEEAVKGLTSLTQLSITYCKRLKCLPEGLQQLTNLSVRYCPTLAKRCEKGIGQDWYKIAHIPHLLITD

(19) United States
(12) Patent Application Publication
(10) Pub. No.: US 2026/0101857 A1
(43) Pub. Date: Apr. 16, 2026

(54) METHOD FOR SELECTING AN S. LYCOPERSICUM PLANT TOLERANT TO HIGH TEMPERATURE CONDITIONS

(71) Applicant: Enza Zaden Beheer B.V., Enkhuizen (NL)

(72) Inventors: Louison Eliane Liard, Amsterdam (NL); Albert Grit, Ermelo (NL); Nejra Solo, Alkmaar (NL); Cornelis Walter Verweij, Enkhuizen (NL)

(21) Appl. No.: 19/116,095

(22) PCT Filed: Sep. 26, 2023
(86) PCT No.: PCT/EP2023/076581

§ 371 Date: Mar. 27, 2025

(30) Foreign Application Priority Data
Sep. 27, 2022 (NL) 2033161

(51) Int. Cl. A01H 1/04; A01H 1/00; A01H 5/08; A01H 6/82

(52) U.S. Cl. CPC A01H 1/045; A01H 1/121; A01H 1/126; A01H 5/08; A01H 6/825

(57) ABSTRACT

The present invention relates to methods for selecting a S. lycopersicum plant that is tolerant to high temperature conditions and has an agronomical phenotype, wherein the plant is absent of an AP locus. The methods furthermore comprise the selecting a plant that is resistant to Tomato Brown Rugose Fruit Virus (TBRFV) comprising a TBRFV resistance gene. Furthermore, the invention relates to high temperature tolerant tomato plants (S. lycopersicum) that show an agronomical phenotype and in addition are preferably resistant to TBRFV.

The present invention relates to methods for selecting a S. lycopersicum plant that is tolerant to high temperature conditions and has an agronomical phenotype, wherein the plant is absent of an AP locus. The methods furthermore comprise the selecting a plant that is resistant to Tomato Brown Rugose Fruit Virus (TBRFV) comprising a TBRFV resistance gene. Furthermore, the invention relates to high temperature tolerant tomato plants (S. lycopersicum) that show an agronomical phenotype and in addition are preferably resistant to TBRFV.

Tomato is one of the most consumed vegetables and economically important crops in the world. Tomato varieties cultivated in different parts of the world change according to cultural and gastronomical factors but also for their adaptation to climate and resistance to pests. The main agricultural traits used to improve in tomato and other crops have been yield, quality, tolerance or resistance to stresses. These traits however result from interactions of several genes (often involved in quantitative trait locus) and the environment. Breeding of new cultivars, where wild relative species of tomato have been used as donors of some traits, are often directed at increasing disease resistance and the domestication of tomato. However, these efforts also resulted in a strong reduction of the genetic diversity of the species and resulted in the loss of alleles of interest for breeding, including trait related to optimal fruit quality, firmness of the fruits, stress tolerance (heat, drought, etc.) and durable resistance to pests and disease in plants.

Segregation distortion or skewed segregation is a naturally occurring phenomenon observed in crop breeding, including tomatoes, in which the genotype/alleles in the progeny of a cross between two varieties or species deviate from expected Mendelian ratios. A segregation distortion gene produces a distortion in normal segregation, so that chromosomes bearing this gene are over- or underrepresented in the progeny. Skewed segregation in a mapping population can be observed due to genetic or environmental factors, or as a result of both. Some of the genetic factors include differences in the chromosome structure or allelic incompatibility, while environmental factors include for example the effect of the environment (such as heat stress, drought, disease pressure, etc.) on the selection pressure of the expression of alleles.

Skewed segregations are frequent events in segregating populations derived from different interspecific crosses in tomato. In tomato, it is known that the rate at which segregation distortion occurs is correlated with increasing distances between the crossing species on the phylogenetic tree. The greater the distance, the greater the distortion in the species. It was observed in a variety of crosses, including S. arcanum with S. lycopersicum, Fulton et al. (1997), S. pennellii with S. lycopersicum (de Vicente and Tanksley, 1993), or S. habrochaites with S. lycopersicum (Bernacchi and Tanksley, 1997). Mating systems in tomato species can range from self-compatible to self-incompatible, or facultative self-compatible/self-incompatible. In most crosses where skewed segregation is observed it has been linked to either self-incompatibility or unilateral incompatibility. In self-incompatibility case, plant rejects pollen from itself preventing inbreeding in that way. In case of unilateral incompatibility, pollen is rejected (even between closely related species) when self-compatible male plant is crossed with self-incompatible female plant.

Tobamovirus is a genus in the virus species Virgaviridae that infects plants, including plants of the Solanaceae family, such as tobacco, potato, tomato, and eggplant and are among the most serious threats to vegetable crops in the world. Tobamoviruses, including the recently newly discovered Tomato Brown Rugose Fruit Virus (TBRFV) are particularly a problem in tomato crops grown in protected environments. TBRFV is transmitted over long distances through external seed contamination, and mechanically from plant to plant through common culture practices through workers' hands, clothes, tools. The virus is capable to preserve infectivity in seeds and contaminated soil for long periods. Furthermore, common weeds, often asymptomatic when infected by the virus comprise a cryptic reservoir between growth cycles. TBRFV infection is associated with necrotic lesions on leaves and tomato plants show mild foliar symptoms at the end of the season but strong brown rugose symptoms on fruits, making the fruit unsuitable for consumption.

In some of the tomato lines derived from crossing of a Tobamovirus resistant (more specifically TBRFV resistant) source, i.e. S. habrochaites, with the elite tomato lines (S. lycopersicum) aberrant (or offtype) phenotypes at heat stress conditions (>28° C., often above 30° C. at night (˜6 h of darkness) and above 36° C. at daytime (˜18 h of light)) were observed, showing deformed and thinned stiff leaves, that can curl. Leaves can also show white stripes and/or yellowing. Plants appear weaker with a delay in development which may also be associated with a poorer setting of the fruit. This aberrant phenotype is apparent at the indicated elevated temperatures at least after 2 weeks of this heat stress and seems to be the result of a strong preference for the S. habrochaites allele (offtype) over the S. lycopersicum allele (non-offtype). Furthermore, in screening a population of up to 450 plants a low number of heterozygous plants was recovered, while the rest of the plants contained the offtype allele. This skewed segregation makes it difficult to remove the offtype region in some of the backgrounds. The understanding of mating barriers between the wild accessions and cultivated tomato, as well as the genetic variability, is important for the improvement of tomato cultivars. Furthermore, it is of great importance for breeders to be able to select donor plants that contain the TBRFV resistance gene, i.e. are resistant to TBRFV, and that do not show the aberrant phenotype and comprises an optimal agronomical phenotype in view of plant vigor, health and fruit production.

Considering the above, there is a need in the art for tomato plants, more specifically S. lycopersicum plants having and maintaining improved agronomical traits at high temperature conditions, i.e. when plants are subjected to elevated temperatures>28° C., even more above 30° C. at night (˜6 h of darkness) and above 36° C. at daytime (˜18 h of light) for a time period of at least two weeks, while at the same time are TBRFV resistant. In addition, there is a need in the art to provide methods for providing TBRFV resistant S. lycopersicum plants having improved agronomical traits and that do not show an aberrant phenotype at such high temperature conditions, wherein the genetics that results in the aberrant phenotype is excluded from the genome of the plant.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a method for selecting a S. lycopersicum plant that is tolerant to high temperature conditions, having an agronomical phenotype, wherein the method comprises the steps of;
a) screening of S. lycopersicum plants for the presence of an AP locus, wherein the presence of the AP locus on (chromosome) chr 9 is identified by the presence of one or more of an “T” on position 379858 in combination with a “C” on position 784380, preferably the presence of a “T” on position 428480 in combination with a “G” on position 601416, more preferably the presence of a “G” on position 474095 in combination with a “T” on position 571352,
b) selecting the S. lycopersicum plants that do not comprise the AP locus, wherein said high temperature conditions is defined as;
   keeping said plant at a temperature of least 28° C. for at least 7 days, preferably for at least 14 days, preferably at least 30° C. at night for about 5 to 7 hours, and at least above 36° C. at daytime for about 17 to 19 h.
      Said improved agronomical phenotype as referred to herein refers to one or more of an increased growth and/or development rate of the plant, absence of leaf deformation, increased plant vigor, increased leaf coverage and/or improved fruit setting, in comparison with plants that do show the aberrant phenotype due to the presence of the AP locus in their genome.

To improve tomato cultivars and overcome the obtained aberrant phenotype often observed in Tobamo resistant and domesticated tomato plants, the implementation of genetic maps and marker assisted selection of QTLs is crucial. Genome mapping has led to the identification of a genetic region or locus controlling physiological disorder triggered by heat stress in tomato. The physiological disorder is hereafter also referred to as ‘aberrant phenotype’ of the tomato plant. The aberrant phenotype locus (AP locus) is located on chromosome 9, originating from S. habrochaites. Furthermore, the AP locus can be co-inherited with a previously identified TBRFV resistance gene located on chromosome 8 in tomato and which shows segregation skewness in favor of the occurrence of the S. habrochaites allele. The present invention furthermore provide a method to uncouple the aberrant phenotype from the TBRFV resistance phenotype. Depending on the genetic background, the aberrant phenotype can be crossed out once inherited, via marker assisted crossing and selection. More specifically, the present invention allows for marker-assisted selection against the AP locus, so that only the desired ToBRFV resistant plants without showing physiological disorders at the indicted elevated temperatures, are obtained.

The aberrant phenotype observed, also indicted herein as physiological disorder, is triggered by the high temperature conditions, i.e. cultivating the seedlings at temperatures of about >28° C., preferably 30° C. or more, preferably at least 36° C., for at least one week, more preferably at least two weeks. These indicated temperatures should be maintained day and night, i.e. including the high temperature of at least 28° C., preferably at least 30° C. at night for about 6 hours and preferably at least 36 C at daytime for about 18 hours, in order to trigger heat stress in the plant and resulting in the aberrant phenotype. When the heat stress is present, the aberrant phenotype has been observed at seedling stage from the moment the plants develop first true leaves, as well as on adult plants. The aberrant phenotype is seen as deformed and thinned stiff leaves, that can curl. Leaves can show white stripes and/or yellowing. Plants appear weaker and with a delay in development. In seedling stage in particular, the true leaves appear stunted. In adult plants in particular, the aberrant phenotype will also be associated with a poorer setting of the fruit.

According to a preferred embodiment, the present invention relates to the method for selecting a S. lycopersicum plant that is tolerant to high temperature conditions, wherein the method in addition provides a S. lycopersicum plant that is resistant to TBRFV, wherein the method further comprises the step of screening and selecting a plant that is resistant to Tobamovirus, wherein said selection comprises establishing the presence of a TBRFV resistance gene comprising a coding sequence having at least 90% nucleotide sequence identity with SEQ ID No. 1. The TBRFV resistance gene is heterozygous or homozygous present in the genome of said plant, preferably homozygous. From the experimental data it can be concluded that the resistance is dominant and that the TBRFV resistance gene and/or genomic sequence must be at least heterozygous present in the genome of the plant to provide resistance against the Tobamo virus. Tomato plant that are resistant to TBRFV comprise the gene conferring resistance, as previously described in WO2020147921, encoded by the coding DNA sequence of SEQ ID No. 1 encoding the protein of SEQ ID No. 2.

According to another preferred embodiment, the present invention relates to the method, wherein after step b) a first S. lycopersicum plant is selected that is resistant to Tobamovirus and is crossed with a second S. lycopersicum plant that is not resistant to Tobamovirus, and subsequently selecting S. lycopersicum plants that are resistant to Tobamovirus.

According to another preferred embodiment, the present invention relates to the method for selecting a S. lycopersicum plant that is tolerant to high temperature conditions, wherein the absence of the AP locus provides an improved agronomical phenotype for said plants at the high temperature conditions in comparison to a S. lycopersicum plant wherein said AP locus is present in its genome, at said high temperature conditions.

According to yet another preferred embodiment, the present invention relates to the method, wherein the S. lycopersicum plant has an improved agronomical phenotype at high temperature conditions of at least 28 C for at least 7 days, in comparison to a S. lycopersicum plant wherein said AP locus is present in its genome, at said high temperature conditions.

According to another preferred embodiment, the present invention relates to the method, wherein the S. lycopersicum plant comprises a TBRFV resistance gene that encodes for a TBRFV resistance protein, wherein the protein has at least 90%, preferably at least 95%, more preferably at least 98%, even more preferably at least 99%, most preferably 100% amino acid sequence identity with SEQ ID No. 2.

The present invention, according to a second aspect, relates to an S. lycopersicum plant that is tolerant to high temperature conditions, wherein the plant is absent of an AP locus on chr 9 in its genome, wherein presence of the AP locus is identified by the presence of one or more of an “T” on position 379858 in combination with a “C” on position 784380, preferably the presence of a “T” on position 428480 in combination with a “G” on position 601416, more preferably the presence of a “G” on position 474095 in combination with a “T” on position 571352.

According to another preferred embodiment, the present invention relates to the S. lycopersicum plant, wherein said plant has an improved agronomical phenotype at high temperature conditions of at least 28 C for at least 7 days, in comparison to a S. lycopersicum plant wherein said AP locus is present in its genome, at said high temperature conditions.

According to yet another preferred embodiment, the present invention relates to the S. lycopersicum plant, wherein the high temperature conditions are maintained for at least 14 days, and wherein the high temperature conditions are at least 30° C. at night for about 5 to 7 hours and at least above 36° C. at daytime for about 17 to 19 h.

According to a preferred embodiment, the present invention relates to the S. lycopersicum plant, wherein the plant is resistant to Tobamovirus, and wherein the plant comprises a TBRFV resistance gene that encodes for a TBRFV resistance protein, wherein the protein has at least 90%, preferably at least 95%, more preferably at least 98%, even more preferably at least 99%, most preferably 100% amino acid sequence identity with SEQ ID No. 2. It is predicted that the TBRFV resistance gene encodes for a NBS-LRR resistance protein. The presence of the resistance gene will decrease the chances of the pathogen overcoming the resistance, or when combined with other resistance genes, disease resistance may even be further improved.

According to another embodiment, the present invention relates to the S. lycopersicum plant, wherein the TBRFV resistance gene comprises a coding sequence that has at least 90% preferably at least 95%, more preferably at least 98%, even more preferably at least 99%, most preferably 100% nucleotide sequence identity with SEQ ID No. 1.

According to a preferred embodiment, the present invention relates to the S. lycopersicum plant, wherein the Tobamovirus is Tomato Brown Rugose Fruit Virus (TBRFV). The present invention, according to a further aspect, relates to plants, plant parts, tissues, cells, and/or seeds derived from the plant or plant obtainable from the method as described above.

The present invention will be further detailed in the following examples and FIGURES wherein:

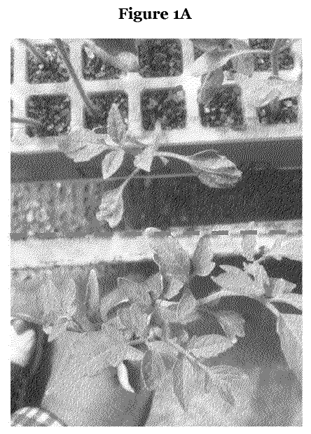

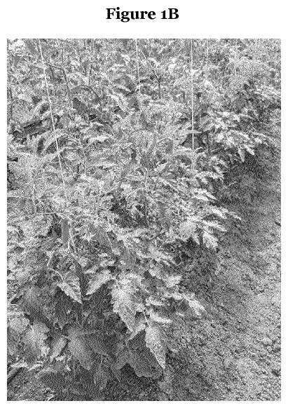

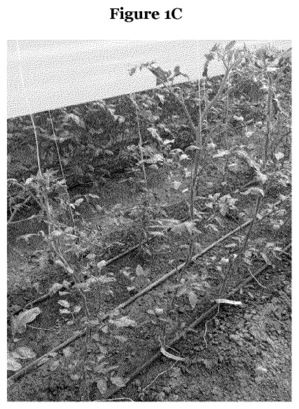

FIG. 1: Shows tomato plants (S. lycopersicum) grown at heat stress conditions (>30° C.) in a greenhouse. These tomato plants have been tested to be resistant to TBRFV and comprise the TBRFV resistance gene, as previously described in WO2020147921. The plants of FIGS. 1A, B, and C are at the same stage of development and growth. Seedlings (FIG. 1A, lower plant) and plants (FIG. 1B) that do not comprise the AP locus do not show the aberrant phenotype. Seedlings (FIG. 1A, upper plant) and plants (FIG. 1C) that are comprised of the AP locus homozygous in their genomes, show the aberrant phenotype, typically presented by curly, deformed, thinned and/or stunted true leaves. Furthermore, plant development is delayed, and leaf coverage and plant vigor are reduced. Several leaves also show yellowing.

### EXAMPLES

### Heat Stress Protocol in Tomato Plants—Aberrant Phenotype Observed During High Temperatures.

Seeds of the mapping population (disclosed above) were sown in soil trays and placed in a growing chamber at the conditions: 18 hours of light (day)/6 hours of darkness (night); with 36° C. during day conditions, and 30° C. during night conditions. Relative humidity was kept above 70%. As control plants, similar tomato plants were used having a similar genetic background to the mapping population but differing in the presence of the AP locus. Below 28° C. no aberrant phenotype was not observed in any of the plants tested.

Seedlings phenotype was scored by visual inspection, 3 weeks after sowing. The presence of the aberrant phenotype was observed as curly, deformed, thinned and/or stunted first true leaves (See FIG. 1A, upper plant). Leaves may also show white stripes and/or yellowing. The plants with the aberrant phenotype appear weaker and with a delay in development (FIG. 1C). Absence of the aberrant phenotype was observed and scored as normal seedling development, as compared to the control. FIG. 1A, lower plant, and FIG. 1B, shows examples of seedlings and plants that are not affected, showing normal development and phenotype. The genotypes of the plants showing the different phenotypes were determined by marker analysis and it was determined that the AP locus, either homozygous of heterozygous, was present in the plants showing the aberrant phenotype and absent in the plants showing the agronomical phenotype. Plants absent of the AP locus showing the agronomical phenotype showed one or more of an increased growth and/or development rate of the plant, absence of leaf deformation, increased plant vigor, and increased leaf coverage in comparison with plants that comprise the AP locus in their genome.

### Fine Mapping and Genome Sequencing of a Tomato Plant Showing Aberrant Phenotype.

Genomic DNA was isolated from TBRFV resistant plants (S. lycopersicum) wherein the aberrant phenotype was present or absent, i.e. comprising the AP locus locus, according to the protocol as published on 27 Apr. 2018 in Nature, Protocol Exchange (2018), Rachael Workman et al., “High Molecular Weight DNA Extraction from Recalcitrant Plant Species for Third Generation Sequencing”. The sequencing libraries were prepared using the PCR free, no multiplex, DNA Ligation Sequencing Kit-Promethion (SQK-LSK109). The isolation procedure resulted in high quality sequencing libraries to be used in the Oxford Nanopore system for sequencing (ONT sequencing). Promethion Flowcell Packs (3000 pore/flowcell) version R9.4.1. were used for sequencing. To resolve the AP locus and further identify the gene(s) providing the aberrant phenotype ONT sequencing was done.

The sequence comparison showed that TBRFV resistant tomato plants with aberrant phenotype comprised on the top of chr 9 a specific region which originates from the S. habrochaites source, which was used as a donor plant providing TBFRV resistance, whereas in the plants that do not show the aberrant phenotype this region on the top of chr 9 was absent. This region was identified as the AP locus and is about 300 Kbp in size, comprising the 48 putative genes. For the fine mapping several markers were developed for genotyping, of which it was found that the about 300 Kbp AP locus was located between markers M6 and M14, more specifically M8 and M11, even more specifically M9 and M10. On the basis of specific SNP the absence or presence of AP was determined in view of the S. lycopersicum Heinz 4.0 genome (also known as SL4.0 genome) and S. habrochaites genome, chr 9.

Table 1 shows the SNP positions and the specific SNP on that position used to determine if the AP locus was absent or present. Using the SNP position and their corresponding SNP sequences tomato plants can be screened and selected for the absence or presence of the AP locus on chr 9. Therefore, tomato plants showing normal growth and development at heat stress conditions, i.e. that do not show the aberrant phenotype at heat stress conditions, can be screened and selected. It was determined that when on position 379858 an “T” and on position 784380 a “C” was present on chr 9, the plant comprised the AP locus and showed the aberrant phenotype. More preferably in case a “G” was present on position 474095 in combination with a “T” present on position 571352 on chr 9, the plant comprised the AP locus and showed the aberrant phenotype.

TABLE 1
Identifying the AP locus in Tobamo (TBRFV)
resistant tomato plants on SNP data

| | Position on SL4.0, | | Lycopersicum allele |
|---|---|---|---|
| Marker | Chr 9 | Habrochiates allele | (SL4.0) |
| M6 | 379858 bp | T | C |
| M8 | 428480 bp | T | C |
| M9 | 474095 bp | G | A |
| M10 | 571352 bp | T | A |
| M11 | 601416 bp | G | A |
| M14 | 784380 bp | C | T |

Inoculation of a Tomato Plant with TBRFV and Determination of TBRFV Infection and Resistance.

The TBRFV isolate AE050 (Origin Saudi Arabia) was used to perform the disease assays. As plant material, the Line OT9, which is a plant line susceptible for TBRFV was used for virus maintenance. Symptomatic leaves received from the original samples were used for sap-mechanical inoculation on the Line OT9. The virus was maintained on systemically infected tomato plants OT9 by monthly sap-mechanical inoculation on new 3 weeks-old seedlings. The tomato plants (S. lycopersicum) were infected with TBRFV isolate AE050 and phenotyped by visual scoring of the plants and the leaves, as described previously in WO2020147921. Visual scoring was performed on a weekly basis. The presence of yellowing, mosaic pattern on leaves, leaf deformation (narrowing, mottling) was recorded on a weekly basis at the plant level. First symptoms were typically observed 12-14 days post-inoculation. Plants were categorized as “resistant” when no such symptoms on leaves were observed. Plants displaying any of the symptoms on leaves were categorized as “susceptible”.

Approximately three weeks after TBRFV inoculation the plants were phenotyped by observation, and ELISA and/or qPCR was performed on lead samples to monitor virus infection as described previously in WO2020147921. Plant that are resistant to TBRFV comprise the gene conferring resistance, as previously described in WO2020147921, encoded by the coding DNA sequence of SEQ ID No. 1 encoding the protein of SEQ ID No. 2.

1. A method for selecting an S. lycopersicum plant that is tolerant to high temperature conditions, having an agronomical phenotype, wherein the method comprises the steps of;
a) screening of S. lycopersicum plants for the presence of an AP locus on chr 9, wherein the presence of the AP locus is identified by the presence of one or more of an “T” on position 379858 in combination with a “C” on position 784380, preferably the presence of a “T” on position 428480 in combination with a “G” on position 601416, more preferably the presence of a “G” on position 474095 in combination with a “T” on position 571352,
b) selecting the S. lycopersicum plants that do not comprise the AP locus,
wherein said high temperature conditions is defined as;
keeping said plant at a temperature of least 28° C. for at least 7 days, preferably for at least 14 days, preferably at least 30° C. at night for about 5 to 7 h, and at least above 36° C. at daytime for about 17 to 19 h,
wherein the method in addition provides a S. lycopersicum plant that is resistant to TBRFV, wherein the method further comprises the step of screening and selecting a plant that is resistant to Tobamovirus, wherein said selection comprises establishing the presence of a TBRFV resistance gene comprising a coding sequence having at least 90% nucleotide sequence identity with SEQ ID No. 1.

2. The method according to claim 1, wherein the absence of the AP locus provides an improved agronomical phenotype for said plants at the high temperature conditions in comparison to a S. lycopersicum plant wherein said AP locus is present in its genome, at said high temperature conditions.

3. The method according to claim 1, wherein the S. lycopersicum plant has an improved agronomical phenotype at high temperature conditions of at least 28 C for at least 7 days, in comparison to a S. lycopersicum plant wherein said AP locus is present in its genome, at said high temperature conditions.

4. The method according to claim 1, wherein the S. lycopersicum plant comprises a TBRFV resistance gene that encodes for a TBRFV resistance protein, wherein the protein has at least 90% amino acid sequence identity with SEQ ID No. 2.

5. An S. lycopersicum plant that is tolerant to high temperature conditions, wherein the plant is absent of an AP locus on chr 9 in its genome, wherein presence of the AP locus is identified by the presence of one or more of an “T” on position 379858 in combination with a “C” on position 784380, preferably the presence of a “T” on position 428480 in combination with a “G” on position 601416, more preferably the presence of a “G” on position 474095 in combination with a “T” on position 571352.

6. The S. lycopersicum plant according to claim 5, wherein said plant has an improved agronomical phenotype at high temperature conditions of at least 28 C for at least 7 days, in comparison to a S. lycopersicum plant wherein said AP locus is present in its genome, at said high temperature conditions.

7. The S. lycopersicum plant according to claim 5, wherein the high temperature conditions are maintained for at least 14 days, and wherein the high temperature conditions are at least 30° C. at night for about 5 to 7 h and at least above 36° C. at daytime for about 17 to 19 h.

8. The S. lycopersicum plant according to claim 5, wherein the plant is resistant to Tobamovirus, and wherein the plant comprises a TBRFV resistance gene that encodes for a TBRFV resistance protein, wherein the protein has at least 90% amino acid sequence identity with SEQ ID No. 2.

9. The S. lycopersicum plant according to claim 8, wherein the TBRFV resistance gene comprises a coding sequence that has at least 90% nucleotide sequence identity with SEQ ID No. 1.

10. The S. lycopersicum plant according to claim 8, wherein the Tobamovirus is Tomato Brown Rugose Fruit Virus (TBRFV).

11. A plant, a plant part, tissue, cell, and/or a seed derived from a plant or plant obtainable from a method according to claim 1.

12. A plant, a plant part, tissue, cell, and/or a seed derived from a plant according to claim 5.
